(19) United States
(12) Patent Application Publication
(10) Pub. No.: US 2025/0176891 A1
(43) Pub. Date: Jun. 5, 2025

(54) An Electrode and Method to Eliminate EEG Signal Contamination Using a Common Reference Electrode

(71) Applicant: Juan G Ochoa, Daphne, AL (US)

(72) Inventor: Juan G Ochoa, Daphne, AL (US)

(21) Appl. No.: 18/841,094

(22) PCT Filed: Aug. 3, 2023
(86) PCT No.: PCT/US23/71561

§ 371 Date: Aug. 23, 2024

(51) Int. Cl. A61B 5/291; A61B 5/00; A61B 5/31; A61B 5/372

(52) U.S. Cl. CPC A61B 5/291; A61B 5/31; A61B 5/372; A61B 5/7203; A61B 5/7264; A61B 2562/046

(57) ABSTRACT

The present invention is an electrode and method to eliminate electroencephalography (EEG) signal contamination using an additional filtered/neutral common reference electrode. The present invention includes an additional filtered reference electrode for filtering noise contamination from EEG recordings and a method of using the same to improve EEG signals sent, received, and recorded for patient treatment. More particularly, the invention is an apparatus/device for filtering EEG signal contamination using an additional filtered reference electrode with a bandwidth and/or signal wavelength filter incorporated therein. An advantage of the present invention is that it is an apparatus for filtering noise contamination from EEG recordings by using an additional filtered reference electrode with a filter whose limits are set by the signal received which is thus used to improve the quality of EEG recordings and the results read therefore.

### BACKGROUND OF THE INVENTION

### Technical Field

This invention relates generally to an Electroencephalography (EEG) electrode (apparatus/device) and method for EEG signal detection filtering. This invention relates more particularly to an apparatus/device for filtering EEG signal contamination using an additional filtered/neutral common reference electrode with a bandwidth and/or signal wavelength filter incorporated therein.

This invention relates generally to apparatuses and devices for EEG signal contamination filtering. This method also can be used with electrophysiological signals, such as electromyography, nerve conduction studies, polysomnography, magneto electroencephalography, and evoked potentials. This invention relates more particularly to an electrode with incorporated frequency bypass filter to eliminate signal noise (signal contamination).

### Background Art

There are apparatuses for taking EEG recordings with electrodes. There are apparatuses, devices, and methods for filtering signal noise contamination from the EEG recording signals received from electrodes.

All of the present art includes or encompasses the weakness of signal contamination for recordings from electrodes. While there are a variety of methods and devices used for filtering signal noise contamination from an EEG recording signals received from electrodes, there is not an apparatus, device, or method to filter said EEG recording signals based on a filtered received signal wherein the filtering is configured from the signal itself and/or a determinable clinical threshold.

What is not present in the prior art are apparatuses for filtering noise contamination from EEG recordings that use an additional filtered reference electrode with a filter whose limits can be set by any practice standard and can be set by the signal received which is thus used to improve the quality of EEG recordings and the results read therefore.

In light of the foregoing prior art, there is a need for a noise contamination filtering apparatus to more accurately record EEG signals. In light of the foregoing prior art, there is a need for an electrode and method to reduce and/or eliminate EEG signal contamination using a common reference electrode.

### BRIEF SUMMARY OF THE INVENTION

The present invention is an additional filtered reference electrode for filtering noise contamination from the reference in EEG recordings and a method of using the same to improve EEG signals sent, received, and recorded for patient treatment.

Electrophysiology potentials, such as Electroencephalography (EEG), are registered through a differential amplifier. The activity from each electrode in the brain is compared to a reference electrode from a different region of the brain. The resulting waveform is the difference between both electrodes.

In a bipolar montage, comparing contiguous electrodes, the reference is cancelled out and it does not play a significant role in the resulting EEG activity. In a referential montage, the recording electrode from each brain region is compared to a reference. The ideal reference would be the least active electrode to prevent interference with the active electrode. In reality, this reference is subject to frequent noise activity that contaminates the recording and hinders the clarity of the EEG.

A method to create an additional filtered reference electrode (O reference) to improve the quality of Electroencephalography (EEG) recording is disclosed. This method includes building an electrode with incorporated high frequency pass filters that only allow frequencies high above the typical frequencies that are clinically relevant.

According to a first aspect of the invention, there is an electroencephalography (EEG) sensing apparatus having an additional filtered reference electrode for improving the quality of an EEG input signal for an EEG recording comprising: a plurality of EEG electrodes wherein at least one of said plurality of EEG electrodes comprises an EEG sensing electrode; wherein said additional filtered reference electrode comprises a filter operating between three and one hundred fifty microvolts configured for limiting frequency data of said EEG recording; wherein said filter comprises a lower frequency limit determined from a highest frequency or a fastest frequency from said EEG input signal; and wherein said EEG input signal is preprocessed by said additional filtered reference electrode.

According to a second aspect of the invention, there is an EEG sensing apparatus wherein said filter comprises said lower frequency limit determined by said highest frequency or said fastest frequency from said EEG input signal increased or decreased by up to one third of said highest frequency or said fastest frequency.

According to a third aspect of the invention, there is an EEG sensing apparatus further comprising a plurality of additional filtered reference electrodes wherein one of said plurality of additional filtered reference electrodes comprises an EEG reference electrode having a filter for limiting a recorded data below said highest frequency or said fastest frequency for said EEG recording, wherein said recorded data is preprocessed by said additional filtered reference electrode.

According to a fourth aspect of the invention, there is an EEG sensing apparatus having an additional neutral reference electrode for improving the quality of an EEG input signal for an EEG recording comprising: a plurality of EEG electrodes wherein at least one of said plurality of EEG electrodes comprises an EEG sensing electrode; wherein said additional neutral reference electrode comprises a filter operating between three and one hundred fifty microvolts configured for limiting frequency data of said EEG recording; wherein said filter comprises a lower frequency limit determined from a highest frequency or a fastest frequency from said EEG input signal; and wherein said EEG input signal is preprocessed by said additional neutral reference electrode.

According to a fifth aspect of the invention, there is an EEG sensing apparatus wherein said filter comprises said lower frequency limit determined by said highest frequency or said fastest frequency from said EEG input signal increased or decreased by up to one third of said highest frequency or said fastest frequency.

According to a sixth aspect of the invention, there is an EEG sensing apparatus further comprising: a plurality of additional neutral reference electrodes wherein one of said plurality of additional neutral reference electrodes comprises an EEG reference electrode having a filter for limiting a recorded data below said highest frequency or said fastest frequency for said EEG recording, wherein said recorded data is preprocessed by said additional neutral reference electrode.

According to a seventh aspect of the invention, there is a method of purifying an electroencephalography (EEG) output signal for clinical significance comprising: applying an EEG sensing apparatus having at least one EEG electrode and at least one additional filtered/neutral reference electrode to a head of a person, obtaining a first electrophysiology data from said EEG electrode, obtaining a second electrophysiology data from said additional filtered/neutral reference electrode, preprocessing said second electrophysiology data, comparing said first electrophysiology data to said second electrophysiology data, filtering said second electrophysiology data, and recording said first electrophysiology data in an unaltered form.

According to an eighth aspect of the invention, there is a method of purifying an EEG output signal wherein said comparing step further comprises a lower frequency filtering limit determined by a highest frequency or a fastest frequency from said EEG output signal.

According to a ninth aspect of the invention, there is a method of purifying an EEG output signal wherein a filter comprises said lower frequency filtering limit determined by said highest frequency or said fastest frequency from said EEG input signal increased or decreased by up to one third of said highest frequency or said fastest frequency.

According to a tenth aspect of the invention, there is a method of purifying an EEG output signal wherein said filter comprises said lower frequency filtering limit determined by a highest clinically significant frequency or a fastest clinically significant frequency.

An advantage of the present invention is that it is an apparatus for filtering noise contamination from EEG recordings by using a clean reference electrode with a filter whose limits are set by the signal received which is thus used to improve the quality of EEG recordings and the results read therefore.

The invention will now be described, by way of example only, with reference to the accompanying drawings in which:

### BRIEF DESCRIPTION OF THE DRAWINGS

FIG. 1 is a diagram of an electrode and method to eliminate EEG signal contamination from a common reference electrode according to the invention; and

FIG. 2 is a diagram of an electrode and method to eliminate EEG signal contamination from a common reference electrode according to the invention.

### DETAILED DESCRIPTION OF THE INVENTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

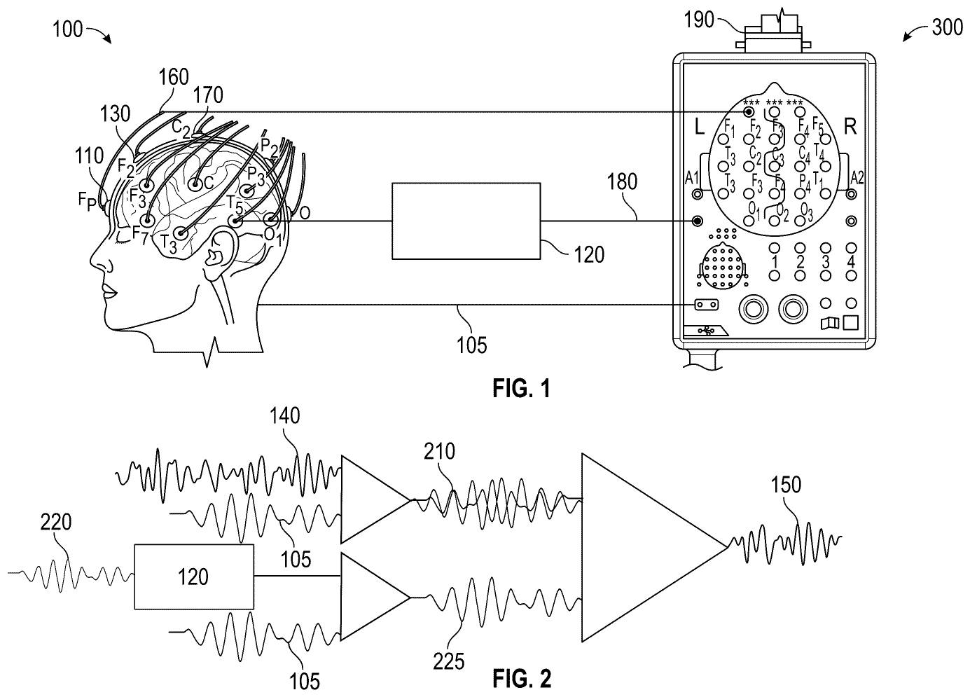

Index of Labelled Features in Figures. Features are listed in numeric order. Referring to the Figures, there is shown in FIGS. 1 and 2 the following features:

Element 100 which is an electrode and method to eliminate EEG signal contamination from an additional filtered/neutral common reference electrode.

Element 105 which is a common reference electrode.

Element 110 which is a reference EEG electrode.

Element 120 which is a filter (bandwidth filter, bandwidth frequency filter, frequency filter, or frequency limiter).

Element 130 which is an EEG electrode.

Element 140 which is an EEG input signal.

Element 150 which is an EEG recording depicting EEG input signal without reference contamination (almost equal to 140).

Element 160 which is an EEG sensing apparatus.

Element 170 which is an EEG sensing electrode.

Element 180 which is an additional filtered reference electrode.

Element 190 which is an electrophysiology data.

Element 200 which is a head of a person.

Element 210 which is a brain activity signal (electronic signal) combined with the common reference activity.

Element 220 which is an activity signal from neutral reference with contamination before filtering.

Element 225 which is a signal after comparing additional filtered/neutral common reference electrode activity and/or a common reference electrode activity.

Element 300 which is a headbox.

The present invention comprises the following two example method and proposed apparatuses and devices.
1. A method, comprising: A recording electrode to use exclusively as a reference to compare with another electrode aiming to obtain electrophysiology data. The reference electrode (hereinafter the O reference) would have a built in filter within the electrode, aimed to remove interference at frequencies below the highest clinically significant frequency.
2. A method, comprising: An input on the headbox to use exclusively as an additional filtered reference. The reference input (O reference) would have a built in filter within the electrode, aimed to remove interference at frequencies below the highest clinically significant frequency.

A headbox is a device used to collect and amplify electrical signals from multiple electrodes placed on a person's scalp. The headbox facilitates the collection, amplification, and preprocessing of electrical signals from multiple electrodes, allowing researchers and clinicians to study brain activity and diagnose various neurological conditions.

The present invention is made by adding a bandpass/frequency filter to an electrode. The filtering/reference electrode is then added to the headbox. The bandpass filter allows only a certain range of frequencies to pass through therefore filtering out signal noise contamination

Filtering the signal noise contamination from the EEG recording signals can be done by using any electrode in an EEG device/apparatus.

The comparator filters by removing the difference between the electrode being read and the reference electrode. In this instance, comparator is defined as an electronic circuit for comparing two electrical signals.

The present invention is used by recording/reading the EEG signal that results from the application of the reference electrode as above.

In an alternative embodiment, there is a clean reference electrode for improving the quality of an EEG recording which includes an EEG electrode. This EEG electrode has a frequency filter for limiting the frequency bandwidth for the EEG recording.

In an alternative embodiment, the frequency filter comprises a frequency limit of a number of hertz, for example the highest clinically significant frequency traditionally used for EEG recordings.

In an alternative embodiment, an EEG sensing apparatus is used for improving the quality of an EEG recording. The recording has clean reference electrodes in which one of the clean reference electrodes has an EEG electrode having a frequency filter for limiting the frequency bandwidth for the recording.

In an alternative embodiment, there is a method of purifying an EEG signal that uses an EEG sensing apparatus with at least one EEG electrode and at least one clean reference electrode attached to the head of a person. Electrophysiology data its then obtained from said EEG electrode and compared to the clean reference electrode. This comparison thus filters the electrophysiology data and records the electrophysiology data as filtered.

In an embodiment, there is an EEG electrode having a built in filter configured to remove interference at frequencies below the highest clinically significant frequency.

In an embodiment, there is a clean reference electrode for improving the quality of an electroencephalography (EEG) recording comprising an EEG electrode having a low frequency filter for limiting frequencies below the highest bandwidth for a recording. Further, there is an alternative version wherein said low frequency filter comprises a frequency limit below the highest clinically significant frequency for EEG recordings, typically 70 Hz for scalp recording and 200 Hz for intracranial recordings.

In an embodiment, there is an electroencephalography (EEG) sensing apparatus for improving the quality of an EEG recording comprising a plurality of clean reference electrodes one of said plurality of clean reference electrodes being a clean reference electrode comprising an EEG electrode having a frequency filter for limiting frequencies below the highest bandwidth for a recording. Further, there is an alternative version wherein said frequency filter comprises a frequency limit below the highest clinically significant frequency for EEG recordings, typically 70 Hz for scalp recording and 200 Hz for intracranial recordings

In an embodiment, there is a method of purifying an electroencephalography (EEG) signal for a clinical significance comprising applying an EEG sensing apparatus having at least one EEG electrode and at least one clean reference electrode to a head of a person, obtaining an electrophysiology data from said EEG electrode, comparing said electrophysiology data to said clean reference electrode thus filtering said electrophysiology data, and recording said electrophysiology data as filtered by the comparing step supra.

In an embodiment of the present invention there is a clean reference electrode for improving the quality of an electroencephalography (EEG) input signal for recording comprising a reference EEG electrode having a filter for limiting a lower frequency limit for a recording. Further, there is an alternative version wherein said filter comprises a lower frequency limit determined by the highest or fastest frequency from said EEG input signal.

In an embodiment, there is an EEG sensing apparatus for improving the quality of an EEG recording comprising a plurality of clean reference electrodes wherein one of said plurality of clean reference electrodes comprises an EEG reference electrode having a filter for limiting below the highest frequency for said EEG recording. Further, there is an alternative version wherein said filter comprises a low frequency limit determined by a highest or fastest frequency from an EEG input signal.

In an embodiment, there is a method of purifying an EEG signal for a clinical significance comprising applying an EEG sensing apparatus having at least one EEG electrode and at least one clean reference electrode to a head of a person, obtaining an electrophysiology data from said EEG electrode, comparing said electrophysiology data to said clean reference electrode thus filtering said electrophysiology data, and recording said electrophysiology data as filtered by the comparing step supra. Further, there is an alternative version wherein said comparing step further comprises a low filtering frequency limit determined by a highest or fastest frequency from said EEG input signal.

According to a preferred embodiment, there is an electroencephalography (EEG) sensing apparatus having an additional filtered reference electrode for improving the quality of an EEG input signal for an EEG recording comprising: a plurality of EEG electrodes wherein at least one of said plurality of EEG electrodes comprises an EEG sensing electrode; wherein said additional filtered reference electrode comprises a filter operating between three and one hundred fifty microvolts configured for limiting frequency data of said EEG recording; wherein said filter comprises a lower frequency limit determined from a highest frequency or a fastest frequency from said EEG input signal; and wherein said EEG input signal is preprocessed by said additional filtered reference electrode.

According to an alternate embodiment, there is an EEG sensing apparatus wherein said filter comprises said lower frequency limit determined by said highest frequency or said fastest frequency from said EEG input signal increased or decreased by up to one third of said highest frequency or said fastest frequency.

According to an alternate embodiment, there is an EEG sensing apparatus further comprising: a plurality of additional filtered reference electrodes wherein one of said plurality of additional filtered reference electrodes comprises an EEG reference electrode having a filter for limiting a recorded data below said highest frequency or said fastest frequency for said EEG recording, wherein said recorded data is preprocessed by said additional filtered reference electrode.

According to a preferred embodiment, there is an EEG sensing apparatus having an additional neutral reference electrode for improving the quality of an EEG input signal for an EEG recording comprising: a plurality of EEG electrodes wherein at least one of said plurality of EEG electrodes comprises an EEG sensing electrode; wherein said additional neutral reference electrode comprises a filter operating between three and one hundred fifty microvolts configured for limiting frequency data of said EEG recording; wherein said filter comprises a lower frequency limit determined from a highest frequency or a fastest frequency from said EEG input signal; and wherein said EEG input signal is preprocessed by said additional neutral reference electrode.

According to an alternate embodiment, there is an EEG sensing apparatus wherein said filter comprises said lower frequency limit determined by said highest frequency or said fastest frequency from said EEG input signal increased or decreased by up to one third of said highest frequency or said fastest frequency.

According to an alternate embodiment, there is an EEG sensing apparatus wherein said filter comprises said lower frequency limit determined by said highest frequency or said fastest frequency from said EEG input signal increased or decreased by up to one third of said highest frequency or said fastest frequency.

According to a preferred embodiment, there is a method of purifying an electroencephalography (EEG) output signal for clinical significance comprising: applying an EEG sensing apparatus having at least one EEG electrode and at least one additional filtered/neutral reference electrode to a head of a person, obtaining a first electrophysiology data from said EEG electrode, obtaining a second electrophysiology data from said additional filtered/neutral reference electrode, preprocessing said second electrophysiology data, comparing said first electrophysiology data to said second electrophysiology data, filtering said second electrophysiology data, and recording said first electrophysiology data in an unaltered form.

According to an alternate embodiment, there is a method of purifying an EEG output signal wherein said comparing step further comprises a lower frequency filtering limit determined by a highest frequency or a fastest frequency from said EEG output signal.

According to an alternate embodiment, there is a method of purifying an EEG output signal wherein a filter comprises said lower frequency filtering limit determined by said highest frequency or said fastest frequency from said EEG input signal increased or decreased by up to one third of said highest frequency or said fastest frequency.

According to an alternate embodiment, there is a method of purifying an EEG output signal wherein said filter comprises said lower frequency filtering limit determined by a highest clinically significant frequency or a fastest clinically significant frequency.

An advantage of the present invention is that it offers apparatuses, devices, and methods for filtering noise contamination from EEG recordings using an additional filtered reference electrode with a filter whose limits can be set by any practice standard and can be set by the signal received which is thus used to improve the quality of EEG recordings and the results read therefore.

Practice standards for filtering noise contamination from EEG recordings include as an example an EEG sensing apparatus of applying/using the present invention wherein the frequency filter comprises a frequency limit below the highest clinically significant frequency for EEG recordings, typically 70 Hz for scalp recording and 200 Hz for intracranial recordings.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. An electroencephalography (EEG) sensing apparatus having an additional filtered reference electrode for improving the quality of an EEG input signal for an EEG recording comprising:
a plurality of EEG electrodes wherein at least one of said plurality of EEG electrodes comprises an EEG sensing electrode;
wherein said additional filtered reference electrode comprises a filter operating between three and one hundred fifty microvolts configured for limiting frequency data of said EEG recording;
wherein said filter comprises a lower frequency limit determined from a highest frequency or a fastest frequency from said EEG input signal; and
wherein said EEG input signal is preprocessed by said additional filtered reference electrode.

19. The EEG sensing apparatus of claim 18 wherein said filter comprises said lower frequency limit determined by said highest frequency or said fastest frequency from said EEG input signal increased or decreased by up to one third of said highest frequency or said fastest frequency.

20. The EEG sensing apparatus of claim 18 further comprising:
a plurality of additional filtered reference electrodes wherein one of said plurality of additional filtered reference electrodes comprises an EEG reference electrode having a filter for limiting a recorded data below said highest frequency or said fastest frequency for said EEG recording,
wherein said recorded data is preprocessed by said additional filtered reference electrode.

21. An electroencephalography (EEG) sensing apparatus having an additional neutral reference electrode for improving the quality of an EEG input signal for an EEG recording comprising:
a plurality of EEG electrodes wherein at least one of said plurality of EEG electrodes comprises an EEG sensing electrode;
wherein said additional neutral reference electrode comprises a filter operating between three and one hundred fifty microvolts configured for limiting frequency data of said EEG recording;
wherein said filter comprises a lower frequency limit determined from a highest frequency or a fastest frequency from said EEG input signal; and
wherein said EEG input signal is preprocessed by said additional neutral reference electrode.

22. The EEG sensing apparatus of claim 21 wherein said filter comprises said lower frequency limit determined by said highest frequency or said fastest frequency from said EEG input signal increased or decreased by up to one third of said highest frequency or said fastest frequency.

23. The EEG sensing apparatus of claim 21 further comprising:
a plurality of additional neutral reference electrodes wherein one of said plurality of additional neutral reference electrodes comprises an EEG reference electrode having a filter for limiting a recorded data below said highest frequency or said fastest frequency for said EEG recording,
wherein said recorded data is preprocessed by said additional neutral reference electrode.

24. A method of purifying an electroencephalography (EEG) output signal for clinical significance comprising:
applying an EEG sensing apparatus having at least one EEG electrode and at least one additional filtered/neutral reference electrode to a head of a person,
obtaining a first electrophysiology data from said EEG electrode,
obtaining a second electrophysiology data from said additional filtered/neutral reference electrode,
preprocessing said second electrophysiology data,
comparing said first electrophysiology data to said second electrophysiology data,
filtering said second electrophysiology data, and
recording said first electrophysiology data in an unaltered form.

25. The method of claim 24 wherein said comparing step further comprises a lower frequency filtering limit determined by a highest frequency or a fastest frequency from said EEG output signal.

26. The method of claim 25 wherein a filter comprises said lower frequency filtering limit determined by said highest frequency or said fastest frequency from said EEG input signal increased or decreased by up to one third of said highest frequency or said fastest frequency.

27. The method of claim 25 wherein said filter comprises said lower frequency filtering limit determined by a highest clinically significant frequency or a fastest clinically significant frequency.
